(19) United States
(12) Patent Application Publication
(10) Pub. No.: US 2014/0107431 A1
(43) Pub. Date: Apr. 17, 2014

(54) Systems and Method for Health Screening For Vascular Disease

(71) Applicant: Alfred S. Callahan, III, Nashville, TN (US)

(72) Inventor: Alfred S. Callahan, III, Nashville, TN (US)

(21) Appl. No.: 13/865,783

(22) Filed: Apr. 18, 2013

Related U.S. Application Data
(63) Continuation of application No. 12/928,032, filed on Dec. 1, 2010, now abandoned.
Application No. 12/928,032 is a continuation of application No. 12/460,744, filed on Jul. 22, 2009, now abandoned.
Application No. 12/460,744 is a continuation of application No. 11/183,364, filed on Jul. 15, 2005, now abandoned.
Application No. 11/183,364 is a continuation-in-part of application No. 10/982,182, filed on Nov. 5, 2004, now Pat. No. 7,506,568.
(60) Provisional application No. 60/517,778, filed on Nov. 6, 2003.

(51) Int. Cl. A61B 8/08; A61B 8/00; A61B 8/06

(52) U.S. Cl. CPC A61B 8/5223; A61B 8/06; A61B 8/467
USPC 600/301; 600/443

(57) ABSTRACT

An angiography system and method comprising of an imaging system for imaging arteries, a processor for processing image data for at least one artery of a participant and an output comprising the image of the artery, a reference image for the artery and physiological data and indexes of the participant.

### RELATED APPLICATIONS AND CLAIM TO PRIORITY

This application claims priority and is a continuation of U.S. patent application Ser. No. 11/183,364, filed Jul. 15, 2009, which is a continuation-in-part of U.S. patent application Ser. No. 10/982,183, filed Nov. 5, 2004, which claims benefit of U.S. Provisional Application No. 60/517,778, filed Nov. 6, 2003. The entire disclosures of these applications are specifically incorporated herein by referenced in their entireties.

### BACKGROUND OF THE INVENTION

1. Field of the Invention

The present invention relates to the field of vascular disease, and more particularly, to inexpensively and efficiently screen for vascular and/or heart disease and aid in the diagnosis thereof.

2. Background of the Invention

Heart attacks and strokes are leading killers in the US, with over 1 million suffering heart attacks each year and over 600,000 suffering strokes. Of these, over half of the heart attack victims will die (a quarter of which will die before reaching the hospital), and 166,000 of the stroke victims will die.

Artery disease (e.g., arteriolosclerosis) is the leading cause of strokes and heart attacks, but over 50% of victims display no warning signs prior to an event. Moreover, peripheral artery disease (PAD), which limits blood flow in the legs, is estimated to affect over 10 million Americans. Those having PAD are at a greater risk of heart attack and stroke.

Approximately 42 million people in the US suffer from cholesterol levels greater than 240 mg/dL—which is a major risk factor for coronary heart disease/arteriolosclerosis and stroke. Of people with high blood pressure, 35% are unaware of it and only 27% are on medication to control it.

Although regular doctor visits may be able to diagnose artery disease, it is often overlooked since, as stated above, over 50% of victims display no warning signs. Moreover, most people do not like going to the doctor, for both psychological and financial reasons, as well as the time it takes for an appointment and waiting for results.

Thus, screening programs would be a beneficial way to identify high risk individuals before such individuals become victims of heart attacks and/or stroke. However, in order for such screening programs to be successful they must: be inexpensive, non-invasive, have instant results, provide information to consumers so that they can consult with a physician and seek treatment and provide information to encourage behavior modification.

Currently, however, there are certain obstacles to screening programs:
physicians don't routinely order such tests (physicians reluctant to order tests for a participant with no symptoms);
such screening tests require a visit to a hospital or outpatient facility which requires at least half the day away from work (many patients cannot afford to give up time off from work);
patients usually experience significant wait times prior to being screened;
cost prohibitive to patients and insurance providers (large deductibles and co-pays, with some insurers refusing to cover tests); and
people are generally not proactive towards healthcare needs.

Thus, there exists a need for an inexpensive, time effective screening test(s) for heart attack and stroke.

### SUMMARY OF THE INVENTION

The present invention presents methods and systems which address the drawbacks listed above, and introduce health risk screening services as a preventative, affordable and convenient means for consumers to identify potential health problems. Embodiments of the invention may be established at retail locations, which bring the “screening” to the consumer, or any healthcare facility (e.g., doctors office, hospital, outpatient medical center). In this way, consumers (participants/customers) can accomplish screening for a disease (e.g., arteriolosclerosis) conveniently and quickly (e.g., before or after shopping, entertainment or a meal).

Embodiments of the invention allow image data of a participant's blood vessels as well as other participant data to be processed and assembled into a single, one-page document for output. Such output may be a single-sided or preferably, double-sided, hardcopy document for the participant to take with them after the screening, as well as an electronic copy which may be displayed on a computer monitor or emailed/communicated over a computer network (e.g., the internet) to the participant and/or the participant's physician/hospital/office. In addition to the participant receiving the results of the screening, the participant may also receive published materials at improving their health and lifestyle for improved living, as well as preliminary diagnostic information.

Other embodiments of the invention may include providing graphical representations for data acquired over a period of time for several screenings, to chart improvement or degradation of a condition.

Accordingly, in one embodiment of the present invention, a health screening system is provided which may include one or more of the following: an imaging system for imaging blood vessels of a participant and input means for inputting participant biographical data and/or one or more physiological values related to the health of the participant. A processor may also be included for: processing image data and participant data, calculating physiological indexes for the participant based on at least one of the image data, the participant data and/or the physiological values, and assembling at least a portion of the image data, the participant data, the physiological values and/or the physiological indexes of the participant into a single document capable of being output. The processor may optionally assemble onto the document corresponding normal values for one or more of the physiological values and/or physiological indexes. The system may also include output means for outputting the document.

In another embodiment of the invention, a method for screening the health of a participant is provided and may include one or more of the following steps: providing an angio-scanning system, which may be the system from the previous embodiment, collecting biographical information of the participant, scanning one or more arteries of a participant, collecting at least one of one or more physiological values of the participant, calculating one or more physiological indexes based on the one or more physiological values, assembling information in a single document after screening related to the health of the participant, the information including images of the scanned arteries with an image of a corresponding normal artery in a single document, and a plurality of the collected physiological values and/or calculated physiological indexes. The method may also include optionally assembling for the single document, corresponding normal values for one or more of the physiological values and/or physiological indexes and outputting the single document.

In yet another embodiment of the present invention, a health-screen result document is provided which may include a single page including images of one or more scanned arteries of a screened participant, each image having a corresponding image of a corresponding normal artery and a plurality of the collected physiological values and/or calculated physiological indexes.

Further embodiments of the invention may include computer readable media having computer instructions for performing at least the above method (as well as other methods of the present invention), as well as a computer application program for performing method(s) of the present invention.

A detailed description of the above embodiments, as well as other objects, advantages and embodiments of the invention are set out in the written description below and attached figures.

### BRIEF DESCRIPTION OF THE DRAWINGS

The file for this application contains at least one drawing executed in color. Copies of an patent issuing from the present application with color drawings will be provided by the Office upon request and payment of the necessary fee.

FIG. 1 illustrates a block diagram of an exemplary health screening system according to an embodiment of the invention.

FIG. 2 is one example of single document with the assembled results and comparative information for a participant according to an embodiment of the present invention.

FIG. 3 is one example of additional information for providing on a reverse side of the single document shown in FIG. 2.

### DETAILED SUMMARY OF THE EMBODIMENTS

Embodiments of the present invention include systems and methods for aiding in the diagnosis of vascular disease (for example), and presents integrated vascular/health screening systems and methods. The present invention provides quick and easy screening at a public location at minimal cost. Using an angio-scanning system, images of a participant's blood vessels (e.g. arteries) are taken, and compared to normal, healthy vessels, and assembled onto a single page of output along with other important participant specific physiological values and/or indexes. Accordingly, as a result of the comparison, the participant may seek medical treatment with a licensed healthcare provider (i.e., MD) if the results indicate that the participant has any indication of vascular or other disease.

Physiological values make include blood pressure (at one or more points on the participants body—e.g., one or both arms, one or both legs), body measurements, pulse, heart rhythm type, aorta diameter, peak systolic velocity (PSV) of blood flow in at least one artery and blood vessel wall thickness. Physiological indexes may include body mass index (BMI), body fat calculation, lean muscle calculation, and ankle brachial index (ABI) for one or both legs.

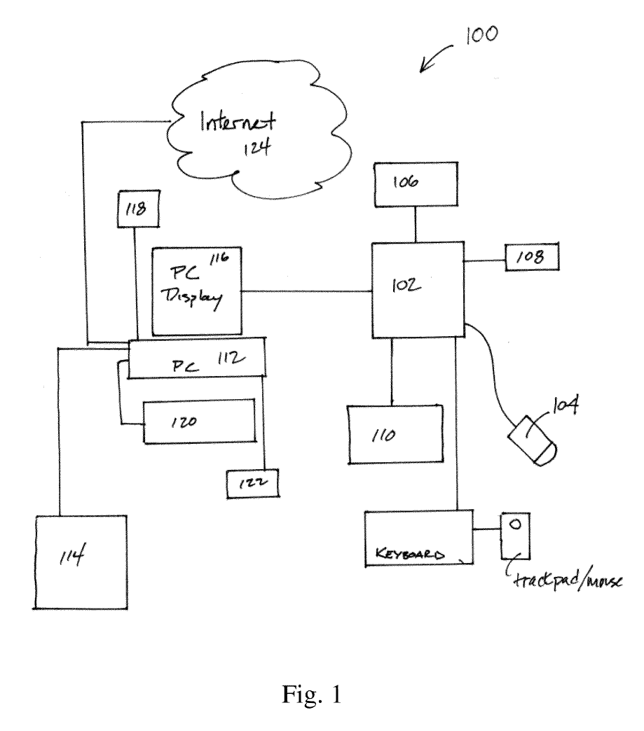

FIG. 1 is a block diagram illustrating an exemplary health screening system 100 according to some embodiments of the present invention. As shown, an ultrasound system 102 includes an ultrasound transducer 104 (e.g., GE® 8L-RS 4-10 Mhz linear vascular transducer, 3C-RS 2-5 Mhz convex abdominal transducer), which transmits a continuous ultrasonic frequency. The frequency is fixed and may be changed (i.e., using a different transducer) based on the depth of the tissue/blood-vessel being examined. Upon the frequency wave encountering tissue, the waves are reflected and received back at the transducer, which produces output voltages based on the reflected waves. The output voltages are then processed into a two dimensional picture.

The ultrasound system may include a display 106, and may also include a speaker 108 and printer 110. Typical ultrasound systems include any known systems familiar to those of skill in the art, for example, Siemens (e.g., SI 400) and General Electric (RT3200, RT3600, RT6800). Preferably, the angiography system is compact, similar to General Electric's LOGIQ Book XP. In addition, the ultrasound system may be similar to those that are disclosed in U.S. Pat. No. 6,108,439, entitled, “Ultrasound image processing system”, issued Aug. 22, 2000, the entire disclosure of which is herein incorporated by reference in its entirety. While the present system is disclosed using ultrasound equipment to image participant tissue, it is understood that other imaging systems (both current and future) are contemplated and may be used in place of or in addition to ultrasound. Such other image scanning equipment may include CAT scan and/or MRI equipment. Preferably, ultrasound equipment is used for cost and/or size considerations.

The ultrasonic frequency that is reflected off a stationary surface maintains the same frequency as that having been transmitted by the transducer. However, the frequency of signals from moving objects (e.g., blood) shifts in proportion to the velocity of the target (Doppler ultrasonography). The shift may be output as an audible signal (via the speaker of the computer and/or ultrasonic system), or, by using spectral analysis, a complete spectrum of frequencies—e.g., blood flow velocities, found in an artery may be determined which can be imaged in different colors/contrasts to depict blood flow in the area of the blood vessel being imaged. The Doppler velocity waveform generally comprises several components: rapid antegrade flow (reaching a peak during systole), transient reversal of flow during early diastole, and slow antegrade flow during late diastole. Generally, the overall blood flow velocity may be referred to in the present invention as peak systolic velocity (PSV). Using such spectral analysis, the severity of a stenosis/plaque in an artery may be better determined. Specifically, at the location of the stenosis, blood velocity usually increases since the area to which the blood flows adjacent the stenosis is smaller.

Other equipment that may be used in addition to the ultrasound/scanning equipment to screen the health of the participant and may include an electrocardiograph (to screen cardiac rhythm and pulse), an ultrasonic Doppler flow detector, a reclining chair or table for the participant to lie down during screening, vascular cuffs, an aneroid sphygmomanometer, electrodes, stethoscope and the like.

Image data as well as other scanning (e.g., ultrasound) data obtained from the ultrasound system may be output (in either digital or analog form; preferably digital), to a computer system 112 (e.g., personal computer) for processing. While the ultrasound system may be directly connected to the computer to transfer the image and other data, the data may be transferred to the computer in any manner known to those of skill in the art (e.g., memory stick, compactflash, smartmedia, disc and the like). The computer may be integrated with the ultrasound system (or may be separately provided as illustrated). The computer may be a personal computer, which may be an Intel® Pentium® based system (or an equivalent thereof) operating Windows® based software (or an equivalent thereof). Connected to the computer may be any of a printer 114, a display/monitor 116, a speaker 118, a keyboard 120 and a mouse 122. In some embodiments of the invention, a software system for receiving and/or processing image data, as well as receiving and/or processing other ultrasound and participant data (e.g., biographical and/or physiological) is operated on the computer. The software may include a graphical user interface (GUI) which allows easy installation, operation, and data entry and analysis/reporting. Operators of the system may include health technicians, screening individuals, administration personnel and others. Data associated with participants (customers) may be added using the GUI. While the present disclosure has presented a stand-alone PC operating software for processing image and other data for screening of a participant, the software may be hosted on a remote server computer and accessed and operated via the internet using a web browser.

Other data from the ultrasound equipment may also be sent to the computer, this may include the participant's pulse, heart rhythm, PSV and blood vessel(s) measurement (e.g., aorta diameter). However, depending upon the ultrasound equipment being used, one or more pieces of such data may not be available from the ultrasound equipment, and thus, may be obtained from other equipment or taken directly by a technician (e.g., pulse) and entered manually into the computer. For example, using an electrocardiograph, the heart rhythm of the participant may be determined (e.g., sinus-normal, A-fib: abnormal). The cardiograph may also be used to obtain the participants pulse. Both pieces of data may be input into the computer.

To that end, any data may be input into the software by manually entry via, for example, the keyboard, mouse or memory devices (CD, other discs, compactflash/smartmedia memory), or by directly connecting other digital electronic medical devices to the computer, so that the data is directly received. With regard to manual entry, data such as the participant's blood pressure, pulse, weight, age, height, medical history, other body measurements (waist, arms, thighs, calves, etc.), body fat measurements, family history, and the like, may be entered. Blood pressure may be measured at the brachial artery (arm) as well as at the ankle, for one or both arms/legs. This allows the calculation of the ABI. The ABI is the ratio of ankle to brachial systolic pressure and is an indicator of arterial blood flow (stenotic) problems. The systolic pressure measured in either or both ankle arteries is used as the numerator in the calculation of the index (for each respective leg), with the denominator being, preferably, the higher of systolic of the brachial/arm pressure: a value of greater than 0.9 is “normal” less than 0.9 is abnormal. Typically, a participant having arterial stenotic problems will have ankle brachial pressure indexes in the range 0.5-0.9, whereas those that are critical, being less than 0.5, whereas those with critical ischaemia usually have an index of <0.5.

The software may also include various reporting and data analysis features on one or more participants. Such features may generate a report which may be displayed on the computer monitor or printed out. Such reports may be summaries of participants, and their corresponding data, and may include charts or graphs to aid in depicting the data.

Typically, a technician may perform an interview with the participant prior to screening to obtain participant data, which may be used to determine what additional information to provide to the participant before and/or after screening. Other questions may be asked post-screening. For example, each of the following questions may be asked and the answer entered into the system.

| | |
|---|---|
| Pre-Screening | |
| Do you have health insurance? | How did you hear about us? |
| Are you taking medications? | How do you prefer to be contacted? |
| Are you a smoker? | If you are diabetic, has there been |
| | any change in glucose control |
| | (hgb AIC)? |
| Are you being treated by a | Where do you fill your prescription |
| physician currently? | medications? |
| Are you seeing a vascular capable | Has this changed (related to above |
| physician? | question)? |
| Do you exercise? | |
| Post-Screening | |
| Did you take your results to your | Have you lost weight since your |
| physician? | screening? |
| What type of physician did you | If yes, how many pounds? |
| see? | |
| Did your doctor order additional | Have you begun an exercise routine? |
| tests? | |
| If yes, what type of tests? | If yes, what does your routine |
| | involve? |
| Did you undergo a procedure? | If you smoked before the screening, |
| | did you continue? |
| If yes, what type of procedure? | Have you altered your diet since the |
| | screening? |
| Did your doctor write you a new | If yes, describe the changes made? |
| prescription? | |
| If yes, what type of prescription/ | On a daily basis, are you more |
| dosage? | health conscientious? |
| Did you doctor modify a current | If yes, how so? |
| prescription? | |
| If yes, what prescription/dosage? | Would you recommend this screening |
| | to a family member or friend? |
| Did your doctor recommend any | If you are a diabetic, has there been |
| over-the-counter (OTC) | any change in glucose control |
| medications? | (hgb AIC)? |
| Post-Screening | |
| If yes, what other OTC | Do you have health insurance? |
| medications were recommended? | |

The participant may also be asked to sign various legal documents (release), or other documents related to the screening. Upon the screening, the participant lies down on a table where one or more technicians conducts the screening. For example, one technician may perform the imaging of the arteries (e.g., carotid, either or both of the internal and common). This technician, or another, may also connect one or more (preferably, 5 or more) electrodes to the participant (e.g., one on the inside of each arm, the inside of each calf and one below the collar bone) from an electrocardiograph machine to conduct an electrocardiogram to determine pulse and/or heart rhythm. Vascular cuffs may be connected to the participant to assess blood pressure, used in combination with a stethoscope, an aneroid sphygmomanometer, and/or an ultrasonic Doppler flow detector, on at least one of the arms and one or both legs for inclusion in the results and/or later computation of the ABI. ABI may be used as an early indicator of heart disease. One or more technicians can collect any other data related to the participant (e.g., weight and/or body measurements for determining, for example, body fat and lean muscle content).

Processing of the data by the computer/software may include assembling image data of one or more scanned arteries (corresponding to one or more images obtained from a participant from the ultrasound apparatus), one or more additional comparative “normal” images of such arteries of the participant subject to ultrasound, and other physiological and biographical participant data (e.g., blood pressure, pulse rate, weight body mass index, age, sex and the like) into a single document, for output (via preferably a color printer) to deliver to the participant when screening is completed. Processing may also include calculation of other physiological values and/or indexes, including blood pressure, pulse, body mass index, ABI for one or both legs, peak systolic velocity (PSV), aorta diameter and the like. Preferably, for example, an image obtained from the ultrasound system of say, for example, the right carotid artery of the participant is formed into an output, with a comparative image of a normal right carotid artery for the participant's age/sex, and with other data (see above). As stated above, this output may be displayed, but is also preferably output to hard-copy via a printer, and preferably printed in several colors to better present the data obtained from the ultrasound apparatus and presented to the participant at the end of the screening.

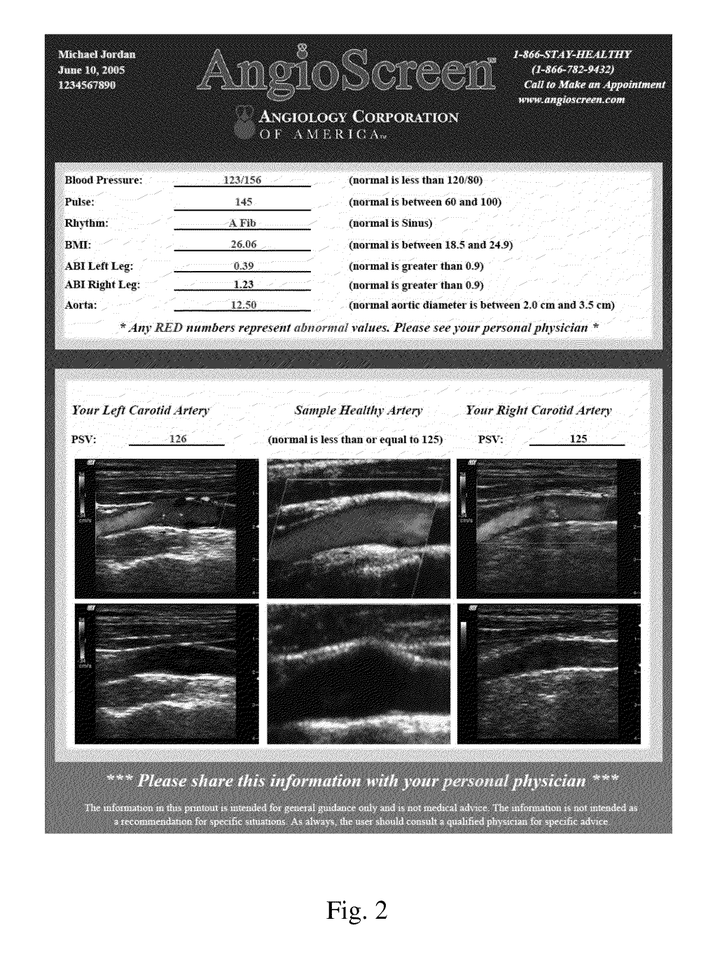

The participant may take the printed output to their doctor for follow-up study. As shown in FIG. 2, which illustrates exemplary printout for the participant, the printout may include the artery images and any one or more of: blood pressure, pulse, heart rhythm classification, body mass index, ankle brachial index of one or both legs, and aorta diameter.

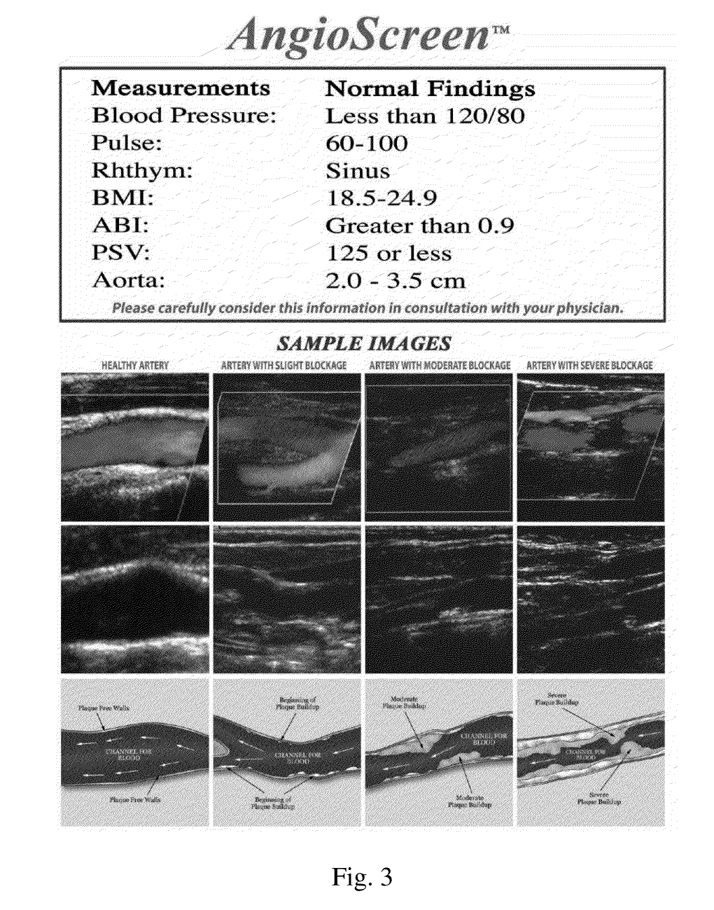

In addition, normal values for each of the above readings may also be printed, which allow the participant to quickly assess abnormal values. Abnormal values may also be more readily identifiable by changing the color, font type, font size, and/or bolding or italicizing of the text. As shown in FIGS. 2 and 3, the abnormal values are red in color, while normal values are black. Such normal values may be:

Blood pressure: Less than 120/80

### Pulse: Between 60-100

### Rhythm: Sinus

### BMI: Between 18.5-24.9

ABI: Greater than 0.90
PSV: 125 or less

### Aorta: Between 2.0-3.5 cm

The bottom section of the printout may include one or more images of blood vessel scans for one or more arteries of the participant having been scanned. For example, as shown, the images comprise three (3) sets of images: one for the left carotid artery, one for a normal artery and one for the right carotid artery. The bottom images represent the actual black and white scans, while the top images may represent the same image with color enhancement which illustrates peak systolic velocity (PSV) measurements.

FIG. 3 represents what may be printed on the reverse side of the printout for the participant. The information that may be included on this statement includes the normal ranges for the values of the information from the first page (FIG. 2). Additional actual sample images of arteries with a range of blockages may also be provided (as well as an image of a healthy artery). Furthermore, illustrations of a blood vessels with varying amounts of plaque may be included, which may correspond to the actual images of the range of blockages (e.g., aligned with the images).

The computer 112 may also be connected to the internet 124, so that the software may be upgraded easily, via download from a website, or for any of the following reasons: data storage and retrieval, communication of results to the participant/physician or hospital/office via email (for example) on the participants home computer or mobile computing/communications device 126.

Other embodiments of the invention may be directed to aspects of doing business. Specifically, one business method embodiment is presented by which a participant has a screening test at a convenient retail (or other public or non-medical facility location), like a pharmacy or department store, and as a result of the screening, may then purchase health products (e.g., vitamins, exercise equipment, diet food, aids, books, and the like). Such a location has been shown to create an additional income for the merchant. In one embodiment, an angio-scanning company provides a system to a merchant, which may include an operator (preferable) or the angio-scanning company may train personnel of the retailer to operate the system. In this aspect, the merchant receives a percentage of the fees charged for the service. In another embodiment, the merchant purchases an angio-screening system and creates a service for which the retailer receives the full fee. In the latter embodiment, the retailer may choose to hire a technician to operate the system.

Having presented the present invention in view of the above-described embodiments, various alterations, modifications, and improvements are intended to be within the scope and spirit of the invention. The foregoing description is by way of example only and is not intended as limiting.

21.-40. (canceled)

41. A health screening system comprising:
an imaging system for imaging blood vessels of a participant;
input means for inputting participant biographical data and/or one or more physiological values related to the health of the participant;
a processor for:
processing image data and participant data,
calculating physiological indexes for the participant based on at least one of the image data, the participant data and/or the physiological values, and
assembling at least a portion of the image data, the participant data, the physiological values and/or the physiological indexes of the participant into a single document capable of being outputted;
assembling for the single document, corresponding normal values for one or more of the physiological values and/or physiological indexes; and
output means for outputting the document, wherein the document further comprises a single page, the single page including images of one or more scanned arteries of a screened participant, each image having a corresponding image of a corresponding normal artery and a plurality of the collected physiological values and/or calculated physiological indexes.

42. The system according to claim 41, wherein the output means is at least one of a display and a printer.

43. The system according to claim 41, wherein the blood vessels comprise arteries.

44. The system according to claim 41, wherein the blood vessels comprise carotid arteries.

45. The system according to claim 41, wherein the input means comprises at least one of a keyboard, a mouse, and a microphone.

46. The system according to claim 41, wherein the participant biographical data comprises at least one of: age, sex, weight, height, pulse and blood pressure.

47. The system according to claim 41, wherein the one or more physiological values is selected from the group consisting of: blood pressure, body measurements, pulse, heart rhythm type, aorta diameter, peak systolic velocity (PSV) of blood flow in at least one artery and blood vessel wall thickness.

48. The system according to claim 41, wherein the one or more physiological indexes are selected from the group consisting of: body mass index, body fat, lean muscle, and ankle-brachial index (ABI) for one or both legs.

49. The system according to claim 41, further including at least one of a computer network and internet connection.

50. A method for screening the health of a participant comprising:
providing an angio-scanning system according to claim 41;
collecting biographical information of the participant;
scanning one or more arteries of a participant;
collecting at least one of one or more physiological values of the participant;
calculating one or more physiological indexes based on one or more physiological values;
assembling information in a single document after screening related to the health of the participant, the information including images of the scanned arteries with an image of a corresponding normal artery in a single document, and a plurality of the collected physiological values and/or calculated physiological indexes;
assembling for the single document, corresponding normal values for one or more of the physiological values and/or physiological indexes; and
outputting the single document, wherein the document further comprises a single page, the single page including images of one or more scanned arteries of a screened participant, each image having a corresponding image of a corresponding normal artery and a plurality of the collected physiological values and/or calculated physiological indexes.

51. The method according to claim 50, wherein the one or more physiological values is selected from the group consisting of: blood pressure, body measurements, pulse, heart rhythm type, aorta diameter, peak systolic velocity (PSV) of blood flow in at least one artery and blood vessel wall thickness.

52. The method according to claim 50, wherein the one or more physiological indexes are selected from the group consisting of: body mass index, body fat, lean muscle, and ankle-brachial index (ABI) for one or both legs.

53. The method according to claim 50, wherein outputting the document comprises at least one of printing the document, storing the document on an electronic medium, or communicating and/or storing the document electronically to a remote location.

54. The method according to claim 50, wherein the information further includes normal values for one or more of the physiological values and/or indexes.

55. The method according to claim 50, further comprising determining a diagnosis of heart disease based on the image data, the physiological values and/or the calculated physiological indexes.

56. The method according to claim 55, further comprising assembling the diagnosis in the single document.

57. The method according to claim 50, further comprising assembling further image information for the document or for a second document, illustrating degrees of severity of arterial blockage.

58. The method according to claim 57, wherein the image information includes actual images and/or illustrations, and wherein the illustrations depict the corresponding plaque buildup within the artery for each degree of severity.

59. A health-screen result document comprising:
a single page, the single page including images of one or more scanned arteries of a screened participant, each image having a corresponding image of a corresponding normal artery and a plurality of the collected physiological values and/or calculated physiological indexes.

60. The system according to claim 59, wherein the information is color coded to alert the participant to abnormal information.
